(19) United States
(12) Patent Application Publication
(10) Pub. No.: US 2014/0135497 A1
(43) Pub. Date: May 15, 2014

(54) SYNTHESIS OF 2,4-DICHLORO-5-TRIFLUOROMETHYL-PYRIMIDINE

(71) Applicants: Li Liu, Shanghai (CN); Deniz Akalay, Frankfurt am Main (DE); Weitong Dong, Shanghai (CN); Jianqing Feng, Suzhou (CN); Christian Wolfgang Hemp, Shanghai (CN); Jun Lu, Shanghai (CN); Le Xie, Shanghai (CN); Jinsong Yang, Ingelheim am Rhein (DE)

(72) Inventors: Li Liu, Shanghai (CN); Deniz Akalay, Frankfurt am Main (DE); Weitong Dong, Shanghai (CN); Jianqing Feng, Suzhou (CN); Christian Wolfgang Hemp, Shanghai (CN); Jun Lu, Shanghai (CN); Le Xie, Shanghai (CN); Jinsong Yang, Ingelheim am Rhein (DE)

(73) Assignee: Boehringer Ingelheim International GmbH, Ingelheim am Rhein (DE)

(21) Appl. No.: 14/072,146

(22) Filed: Nov. 5, 2013

(30) Foreign Application Priority Data
Nov. 13, 2012 (CN) PCT/CN2012/084508

(51) Int. Cl. C07D 239/30; C07D 239/54

(52) U.S. Cl. CPC C07D 239/30; C07D 239/54
USPC 544/309; 544/334

(57) ABSTRACT

This invention relates to a novel method for the synthesis of 2,4-dichloro-5-trifluoromethyl-pyrimidine useful as intermediate in the manufacture of pharmaceutically active ingredients.

### FIELD OF THE INVENTION

This invention relates to a method for the synthesis of 2,4-dichloro-5-trifluoromethyl-pyrimidine useful as intermediate in the manufacture of pharmaceutically active ingredients.

### BACKGROUND OF THE INVENTION

2,4-dichloro-5-trifluoromethyl-pyrimidine (5-TFP) is an important intermediate for the manufacture of pharmaceutically active ingredients, for example for the treatment of cancer as described in WO 2010/055117.

The known chemical syntheses of this intermediate offer only limited accessability of 5-TFP and all face severe drawbacks that do not allow for a sustainable and environmentally friendly supply with this important intermediate. Therefore there was a need to develop a novel approach to this compound. This disclosure describes a novel, environmentally benign and sustainable process for the manufacture of 5-TFP.

The known routes to 5-TFP are described in the following:

5-TFP can be prepared by a two-step process using gaseous CF₃I as a trifluoromethylation reagent (scheme 1) via trifluoromethyl uracil (5-TFU) as described in WO 2007/055170.

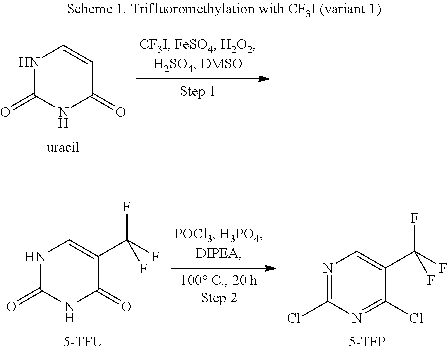 [SMILES: O=c1cc[nH]c(=O)[nH]1 ; O=c1[nH]cc(C(F)(F)F)c(=O)[nH]1 ; FC(F)(F)c1cnc(Cl)nc1Cl]

The disadvantage of this process is the difficult handling of the toxic and expensive gaseous reagent. Additionally, this process has to be run in environmentally inappropriate solvents like dimethylsulfoxide.

A further route of synthesis is disclosed in CN 101955466. Therein, the combination of H₂SO₄ and FeSO₄ of step 1 in variant 1 as depicted in scheme 1 above is replaced by HBF₄/Fe₂(SO₄)₃ (scheme 2), however, all main disadvantages of that route remain.

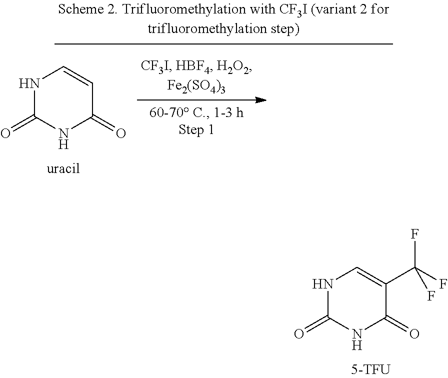 [SMILES: O=c1cc[nH]c(=O)[nH]1 ; O=c1[nH]cc(C(F)(F)F)c(=O)[nH]1]

An alternative process to 5-trifluoromethyl-uracil (5-TFU) as a precursor of 5-TFP is revealed in U.S. Pat. No. 5,352,787 describing a four-step access starting from 5-methyl-uracil (scheme 3). This process suffers from the necessity to use large amounts of two highly toxic and corrosive gases (Cl₂ and HF), which make this process inappropriate for normal pharmaceutical manufacturers and environmentally and from a safety point of view unfavorable.

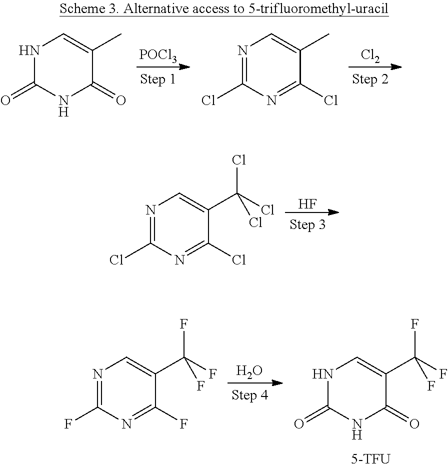

Recently LANGLOIS reagent (sodium trifluoromethanesulfinate, CF₃SO₂Na) was successfully used as reagent in the trifluoromethylation reaction of uracil (PNAS 2011, 14411-14415) under biphasic conditions.

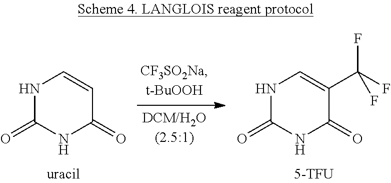 [SMILES: O=c1cc[nH]c(=O)[nH]1 ; O=c1[nH]cc(C(F)(F)F)c(=O)[nH]1]

However, mixtures of chlorinated organic solvent with water or sulfoxides with water are used. These conditions are unsuitable for larger scale application because of the uncontrollable exothermic nature of the reaction, the vigorous stirring needed and the large amounts of chlorinated organic wastes. In addition, the inventors identified a new unknown impurity when a scaled-up prior art trifluoromethylation process was applied to uracil causing additional need for purification. As additional drawbacks inconsistent yields with incomplete conversion of starting material, long reaction times, accumulation of peroxides (also due to high number of equivalents needed) and the need to use lab grade (=expensive) CF₃SO₂Na instead of commercial grade (=cheap; usually only 50-70 pure) CF₃SO₂Na were identified which make the prior art process not feasible for an economically competitive way to produce larger amounts of the title compound.

Thus, it was necessary to investigate a potential new method to address these drawbacks.

### DETAILED DESCRIPTION OF THE INVENTION

After careful investigation of the above prior art methods, the CF₃ transfer reaction using LANGLOIS reagent as shown in scheme 4 was further elaborated:

First of all, when applying the standard procedure disclosed in PNAS to uracil (DCM/water 2:1, 0° C. with lab grade CF₃SO₂Na) complete conversion of starting material was reached after 22 h and 5-TFU was obtained in a yield of 55% (crude product; comparative example 1). A previously unknown impurity with high polarity was found by HPLC during the reaction which might cause problems in any downstream steps.

When this reaction is run in DCM or DMSO on larger scale, a large volume of organic waste is generated. Additionally, the use of organic peroxides in an organic medium poses very high safety constrains since accumulation of these peroxides may lead to explosive mixtures. Thus, a transfer of the trifluoromethylation step to aqueous medium and use of lower amounts of peroxides is desirable and allows for a large reduction of organic waste and safer running conditions, since in water the use of organic peroxides is much safer than in organic media and the exotherm reaction can be controlled more easily.

However, when the standard procedure was adapted to use water only (water, 0° C. with lab grade CF₃SO₂Na) the obtained results worsened significantly. After 24 h the conversion rate only reached 36% (comparative example 2) making this method completely useless. This shows that as far as water-soluble uracil is concerned a simple shift to water (in contrast to the disclosure of PNAS) is not possible on a large scale.

It was very surprising to find out that the results obtained in water could be significantly improved when the reaction mixture is kept at a temperature of about 40-100° C. after the addition of an aqueous solution of the organic peroxide. Reaction time to full conversion can be reduced, total amount of organic peroxide can be reduced, reasonable yields are obtained and build-up of peroxides can be prevented.

Thus, the present invention relates to a method for preparing 5-trifluoromethyl-uracil (5-TFU) comprising

a) trifluoromethylation of uracil with sodium trifluoromethanesulfinate (CF₃SO₂Na) and organic peroxide in water to form 5-trifluoromethyluracil (5-TFU), wherein the reaction temperature after the addition of an aqueous solution of organic peroxide is kept in a range of about 40-100° C.

In addition, the present invention relates to a method for preparing 2,4-dichloro-5-trifluoromethyl-pyrimidine (5-TFP) comprising

a) trifluoromethylation of uracil with sodium trifluoromethanesulfinate (CF₃SO₂Na) and organic peroxide in water to form 5-trifluoromethyluracil (5-TFU), wherein the reaction temperature after the addition of an aqueous solution of organic peroxide is kept in a range of about 40-100° C., and
b) reacting 5-trifluoromethyluracil (5-TFU) with phosphoryl chloride (POCl₃) to form 2,4-dichloro-5-trifluoromethyl-pyrimidine (5-TFP).

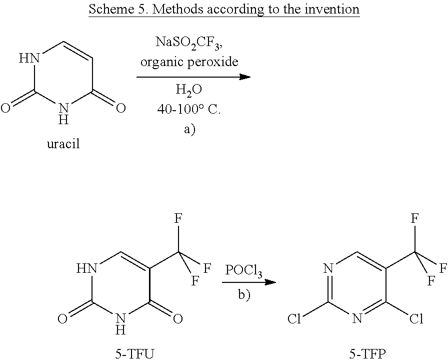

Uracil required as the starting material is commercially available.

The first step according to the method is the trifluoromethylation of uracil with sodium trifluoromethanesulfinate (CF₃SO₂Na) and organic peroxide, e.g. tert-butyl hydroperoxide (TBHP), in water and optionally a transition metal catalyst, e.g. FeSO₄.

Sodium trifluoromethanesulfinate (CF₃SO₂Na) which is also called LANGLOIS reagent, is a safely and easily handable solid and commercially available (usually 50-70% pure).

Commercially available 70% aqueous solution of tert-butyl hydroperoxide (TBHP) is a suitable oxidant for the trifluoromethylation.

All the reagents used in the trifluoromethylation step are soluble in water being a good solvent for this step.

The strongly exothermic nature of this method can be well controlled by adjusting the dosing rate of organic peroxide, e.g. tert-butyl hydroperoxide (TBHP), and/or addition of a transition metal catalyst.

THF, 2-MeTHF, ethyl acetate and isopropyl acetate can be used as extraction solvents in the workup of the reaction step a). THF and 2-MeTHF have advantages in terms of solubility, ethyl acetate and isopropyl acetate however yield slightly higher quality of the produced intermediate (5-TFU).

Alternatively, 5-TFU can be obtained from the reaction mixture by concentration of the aqueous phase and filtration of the precipitate.

If necessary, the intermediate 5-TFU can be further purified by recrystallization from water or isopropyl acetate.

In one embodiment of the methods the sodium trifluoromethanesulfinate (CF₃SO₂Na) used in step a) has a commercial grade of equal to or below 80% sodium trifluoromethanesulfinate (CF₃SO₂Na).

In one embodiment of the methods the sodium trifluoromethanesulfinate (CF₃SO₂Na) used in step a) has a commercial grade of about 50-70% sodium trifluoromethanesulfinate (CF₃SO₂Na).

In a further embodiment of the methods the commercial grade sodium trifluoromethanesulfinate (CF₃SO₂Na) is pre-treated before use in step a).

In a further embodiment of the methods the commercial grade sodium trifluoromethanesulfinate (CF₃SO₂Na) is pre-treated before use in step a) by slurrying in ethyl acetate, filtration and concentration.

In a further embodiment of the methods the commercial grade sodium trifluoromethanesulfinate (CF₃SO₂Na) is pre-treated before use in step a) by slurrying the commercial grade sodium trifluoromethanesulfinate (CF₃SO₂Na) in ethyl acetate, heating of the resulting suspension to about 40-50° C., stirring at this temperature, filtering of the suspension, adding water to the filtrate and removing substantially all ethyl acetate.

One method of pre-treatment of sodium trifluoromethanesulfinate (CF₃SO₂Na) when used as reagent is known from US 2011/0034530. However, the purification method used therein is much more laborious and the type of reaction the reagent is used for is different (sulfinylation).

The pre-treatment of commercial grade sodium trifluoromethanesulfinate (CF₃SO₂Na, 50-70% pure) does not only allow the use of this cheap ingredient but also has significant advantages in terms of possible yields. When commercial grade sodium trifluoromethanesulfinate (CF₃SO₂Na) is used without pre-treatment the yields which could be obtained turned out to be inconsistent and tended to result in lower yields (comparative example 3).

In a further embodiment of the methods the organic peroxide used in step a) is tert-butyl hydroperoxide (TBHP), preferably as an aqueous solution.

In a further embodiment of the methods the organic peroxide used in step a) is tert-butyl hydroperoxide (TBHP), wherein the aqueous solution of tert-butyl hydroperoxide (TBHP) has a content of about 70% tert-butyl hydroperoxide (TBHP).

In a further embodiment of the methods the organic peroxide used in step a) is continuously dosed to the reaction mixture.

In a further embodiment of the methods the organic peroxide used in step a) is used in an amount of about 4 eq. in relation to uracil.

In a further embodiment of the methods the addition rate of the organic peroxide used in step a) is controlled to keep the reaction temperature in a range of about 45-75° C., preferably 45-55° C., during addition.

The control of the addition rate helps to slow the dosage of the organic peroxide and the rate of peroxide decay can be carefully controlled in order to run the method without risking accumulation of peroxides.

In a further embodiment of the methods the reaction temperature in step a) is kept in a range of about 40-100° C. after the addition of an aqueous solution of organic peroxide until the ratio of uracil:5-trifluoromethyl-uracil is equal to or below 3:97.

In a further embodiment of the methods the reaction temperature in step a) is kept in a range of about 40-80° C. after the addition of an aqueous solution of organic peroxide.

In a further embodiment of the methods the reaction temperature in step a) is kept in a range of about 45-70° C. after the addition of an aqueous solution of organic peroxide.

In a further embodiment of the methods the reaction temperature in step a) is kept in a range of about 60-70° C. after the addition of an aqueous solution of organic peroxide.

In a further embodiment of the methods the reaction temperature in step a) is kept in a range of about 45-60° C. after the addition of an aqueous solution of organic peroxide.

In a further embodiment of the methods the reaction in step a) is carried out in the presence of a transition metal catalyst.

In a further embodiment of the methods the reaction in step a) is carried out in the presence of FeSO₄ as catalyst.

The optional addition of a transition metal catalyst, e.g. FeSO₄, further reduces the risk of peroxide accumulation. Without this catalyst, the reaction also works, however, peroxide accumulation may render severe issues of process safety. Therefore the revealed method includes, but does not necessitate, the use of for example a FeSO₄ additive for large scale production.

In a further embodiment of the methods the reaction in step a) is carried out in the presence of silica gel.

The addition of silica gel as HF absorber has the advantage of preventing HF corrosion of the glass lining (HF is formed in the reaction) and of further improving process safety.

In a further embodiment of the methods a solvent selected from among THF, 2-MeTHF, ethyl acetate and isopropyl acetate is used to extract 5-TFU obtained in reaction step a).

In a further embodiment of the methods 5-TFU obtained in reaction step a) is isolated without extraction by concentration of the aqueous phase and filtration.

In a further embodiment of the method the intermediate 5-TFU obtained in step a) is chlorinated with or without isolation of 5-TFU.

In a further embodiment of the method the intermediate 5-TFU obtained in step a) is chlorinated with or without isolation of 5-TFU to 5-TFP in step b) using a mixture of phosphoric acid (H₃PO₄), phosphoryl chloride (POC1₃) and diisopropylethyl amine (DIPEA).

### EXAMPLES

### Example 1

### Preparation of 5-trifluoromethyluracil (5-TFU)

### Pretreatment of Commercial Grade Sodium Trifluoromethanesulfinate (CF₃SO₂Na)

A 1 L jacket reactor (reactor A) is charged with CF₃SO₂Na (125.0 g, ˜65% purity, ˜0.52 mol, 2.89 eq.) followed by ethyl acetate (625.0 g). The resulting suspension is heated to 40-50° C. and kept stirring at this temperature for 1 h. The suspension is filtered with the aid of Celite® (5.0 g) at 30-50° C. and the cake is washed with ethyl acetate (50 g). The combined filtrate is transferred to a 500 mL jacket reactor (reactor B) and concentrated to about 80-100 mL (jacket temperature 70° C./100-500 mbar). Water (100 mL) is added into the mixture. The resulting biphasic mixture is concentrated to about 90 mL (jacket temperature 70° C./100-500 mbar) to remove residual ethyl acetate.

Trifluoromethylation Uracil (20.0 g, 0.18 mol, 1.0 eq.), silica gel (4.0 g), ferrous sulfate (FeSO₄) heptahydrate (2.0 g, 0.007 mol, 0.04 eq.) and water (100.0 mL) are charged into reactor B. The resulting suspension is heated to 45-50° C., tert-butyl hydroperoxide (91.8 g, 0.71 mol, 70% aqueous solution, 3.9 eq.) is added slowly into the mixture through an addition funnel while keeping the internal temperature between 45-55° C. during addition by controlling the addition rate and jacket reactor in about 30 min. A strong exotherm together with release of gas is observed during the addition of the tert-butyl hydroperoxide. A large amount of SO₃ and trace amount of HF are detected. Silica gel is used to minimize the corrosion of the glass reactor. After the addition, the internal temperature is kept to 60-70° C. for 1.0-1.5 h. The reaction is monitored by HPLC until ratio of uracil:5-TFU <3:97 (HPLC area). Aqueous sodium sulfite solution (15 g (15 weight %), 0.0178 mol, 0.1 eq.) is added and stirred for another 30 min between 60-70° C. to quench residual tert-butyl hydroperoxide. The peroxide level is checked with test strip (Merckoquant 110011 test strips until it is lower than 10 ppm). The mixture is then cooled to 25-35° C., diluted with THF (40 mL), the resulting mixture is filtered (to remove silica gel) and the filter cake is washed with THF (20 mL). The combined filtrate is concentrated to about 170-190 mL (jacket temperature 70° C./100-500 mbar). The resulting suspension is cooled to 10-15° C. in 2 h and held at this temperature for 1 h. The suspension is filtered, the filter cake is washed with cold water (20 mL) and dried at 45-50° C. to obtain white crystal (24 g; crude yield 75%, HPLC assay yield is 73%, area purity>97% (220 nm). (Note: The resulting product containing some inorganic impurities was used directly in the following chlorination step. Around 4 g of product is lost in the aqueous mother liquor, which can be recovered by extraction with THF if necessary.) (Note: From a quality point of view the reaction without FeSO₄ and silica gel gives similar results)

### 5-trifluoromethyluracil (5-TFU)

White solid.

¹H NMR (CD₃COCD₃): δ 8.1 (s, 1H), 10.5 (brs, 2H).

¹⁹F NMR (CD₃COCD₃): δ −63.8

ESI MS (m/z) 179 [M−1]⁻

### Example 2

### Preparation of 2,4-dichloro-5-trifluoromethylpyrimidine (5-TFP)

To a jacket reactor (500 mL) is added 5-trifluoromethyluracil (5-TFU, 40 g, ˜70% assay, ˜0.16 mol, 1.0 eq.), H₃PO₄ (2.4 g; 0.02 mol, 0.13 eq.) and POCl₃ (128 g; 0.83 mol, 5.2 eq.) (a white suspension is formed). DIPEA (35 g, 0.27 mol, 1.69 eq.) is added to the suspension dropwise in about 10 min and then the reaction mixture is heated to 110-120° C. (clear solution). The reaction is monitored with HPLC until ratio 5-TFU:5-TFP<5:95 (reaction normally finished in 7-8 h; if reaction is not complete, additional POCl₃ (5 g, 0.032 mol, 0.2 eq) and DIPEA (1.3 g, 0.01 mol, 0.06 eq) are charged and stirred for another 1-2 h). The reaction is then cooled to rt and n-butyl acetate (80 mL) is added to the reaction mixture. About 60 mL of distillate (POCl₃ and some n-butyl acetate) is collected at 63-65° C./450-500 mbar. The resulting dark solution is slowly added to a mixture of conc. HCl (165 g, 27 weight %, 1.23 mol, 7.7 eq.) and methyl tertiary butyl ether (MTBE, 120 mL) while the temperature is maintained below 20° C. The organic phase is separated and the aqueous phase is extracted with MTBE (2×120 mL). The organic phase is gathered, washed with water until the pH value reaches ca. 5-6. MTBE is removed under reduced pressure (˜42° C./200 mbar), the final product is purified through distillation (87-89° C./55 mbar) to afford 5-TFP as colorless oil (25.3 g, yield 72.9%; purity 98%).

### 2,4-dichloro-5-trifluoromethylpyrimidine (5-TFP)

Colorless to light yellow oil

¹H NMR (CD₃COCD₃): δ 8.8 (s, 1 H),

¹⁹F NMR (CD₃COCD₃): δ −63.7

ESI MS (m/z) 216 [M−1]⁻

### Comparative Example 1

### Preparation of 5-trifluoromethyluracil (5-TFU) According to PNAS 2011, 14411-14415 Under Biphasic Conditions

To a mixture of uracil (0.5 g, 4.5 mmol) and sodium trifluoromethanesulfinate (2.1 g, 13.5 mmol, 3.0 eq.) in DCM (18 mL) and water (7 mL) is added dropwise tert-butyl hydroperoxide (70% solution in water, 2.9 g, 22.5 mmol, 5 eq.) with vigorous stirring while controlling the inner temperature around 0-2° C. The reaction is allowed to warm to rt (20-22° C.) and monitored by HPLC until completion (completed in 22 h). The reaction mixture is distilled under vacuum at rt to remove DCM, the resulting mixture is extracted with ethyl acetate (4×20 mL). The combined organic layers are dried with sodium sulfate and concentrated to obtain 5-TFU as white solid (assay yield 55%). Note: A new impurity with high polarity was found under 254 nm by HPLC during the reaction.

### Comparative Example 2

### Preparation of 5-trifluoromethyluracil (5-TFU) According to PNAS 2011, 14411-14415 Under Monophasic (Aqueous) Conditions

To a mixture of uracil (0.5 g, 4.5 mmol) and sodium trifluoromethanesulfinate (2.1 g, 13.5 mmol, 3.0 eq.) in water (25 mL) is slowly added tert-butyl hydroperoxide (70 solution in water, 2.9 g, 22.5 mmol, 5 eq.) with vigorous stirring while controlling the inner temperature around 0-2° C. The reaction is allowed to warm to rt (20-22° C.) and monitored by HPLC. The conversion is only 36% after 24 h.

### Comparative Example 3

### Preparation of 5-trifluoromethyluracil (5-TFU) According to Process of the Invention Without Pre-Treatment of Sodium Trifluoromethanesulfinate (CF₃SO₂Na)

A jacket reactor (2 L) is charged with uracil (50 g, 0.446 mol, 1 eq.), sodium trifluoromethanesulfinate (311.0 g, 65%, 1.293 mol, 2.9 eq.), ferrous sulfate (FeSO₄) heptahydrate (5.0 g) and water (500 mL). The resulting suspension is heated to 40° C. Tert-butyl hydroperoxide (287 g; 70% aqueous solution, 2.232 mol, 5 eq.) is added slowly into the mixture while keeping the internal temperature between 55-75° C. After addition of peroxide, the resulting mixture is stirred between 50-60° C. for 0.5 to 1.0 h. The reaction is monitored by HPLC until the ratio of uracil:5-TFU<3:97 (HPLC area). The peroxide residue is quenched with aqueous sodium sulfite solution until peroxide concentration is below 10 ppm. The resulting mixture is extracted with 2-MeTHF (4×250 mL) and the combined organic phase is washed with NaCl aqueous solution (25%; 150 mL). The organic phase is then concentrated to get the crude product as white solid. HPLC assay yield is 48%.

Note: Direct use of the commercial grade CF₃SO₂Na gives variable yield. In this batch, the assay yield is around 20% lower than the normal result when using the reagent after pre-treatment (see pre-treatment procedure in example 1; i.e. the reaction under the conditions of comparative example 3 with pre-treatment according to example 1 has an HPLC assay yield of 67%). Significant amounts of impurities with high polarity are found in the HPLC monitoring of the reaction (220 nm).

HPLC method

| Equipment | Agilent 1200 series Gradient HPLC apparatus | | |
|---|---|---|---|
| Eluent A | water | | |
| Eluent B | methanol | | |
| Column | Luna 3u phenyl-hexyl 150 × 4.60 mm | | |
| Column | 40° C. | | |
| Temperature | | | |
| Flow | 0.7 mL/min | | |
| Gradient | time (min) | water (%) | methanol (%) |
| | 0.00 | 95 | 5 |
| | 7.00 | 30 | 70 |
| | 12.00 | 20 | 80 |
| | 18.00 | 20 | 80 |

| | |
|---|---|
| Diluent | methanol: H₂O = 1:1 |
| Sample | ca. 25 mg of sample was dissolved in 25 mL methanol/water |
| Preparation | (1:1) |
| Injection | 1 μL |
| Volume | |
| UV Detection | wavelength: 254 nm, 220 nm, bandwidth: 8 nm |
| | reference: off |
| | peakwidth (response time): >0.1 min (6 s) or comparable |
| System | For purity testing of final product the attributes of the |
| Suitability | principal peak should not violate following ranges: |
| | Symmetry factor between 0.8 and 1.5. |
| | Height between 0.8 and 1.2 AU (resp. 0.8 to 1.2 V). |
| | Retention time as stated below ±5%. |

GC method for the final product

| Equipment | Agilent 7891A Gas chromatograph with | | |
|---|---|---|---|
| | flame ionization detector (FID) | | |
| Carrier Gas | Helium (constant flow = 1.5 mL/min) | | |
| Detector Gases | Gas | Flow | |
| | hydrogen | 35 | mL/min |
| | synth. air: | 350 | mL/min |
| | makeup (Helium): | 26.5 | mL/min |

| | |
|---|---|
| Injection Mode | Split 1:20 |
| Injection Volume | 1 μL |
| Diluent | ACN |
| Sample Preparation | Sample was diluted in ACN |
| Column | Type: HP-5.5% phenyl methyl siloxan; L = |
| | 30 m; ID = 0.32 mm; Film = 0.25 μm |
| | Supplier: agilent; Part No.: 19091J-413 |
| Temperature (Injector) | 260° C. |
| Temperature (Detector) | 280° C. |
| Temperature (Oven) | Initial temp.: 50° C. |
| | Hold time: 5 min |
| | Rate: 15° C./min |
| | Final Temp.: 250° C. |
| | Run time = 17 min |
| System Suitability | For purity testing of final product the |
| | attributes of the principal peak should not |
| | violate following ranges: |
| | Symmetry factor between 0.8 and 1.5. |
| | Height between 0.8 and 1.2 AU (resp. 0.8 |
| | to 1.2 V). |
| | Retention time as stated below ±5%. |

1. A method for preparing 5-trifluoromethyl-uracil (5-TFU) comprising
a) trifluoromethylation of uracil with sodium trifluoromethanesulfinate (CF₃SO₂Na) and organic peroxide in water to form 5-trifluoromethyluracil (5-TFU), wherein the reaction temperature after the addition of an aqueous solution of organic peroxide is kept in a range of about 40-100° C.

2. Method according to claim 1 wherein the sodium trifluoromethanesulfinate (CF₃SO₂Na) used in step a) has a commercial grade of about 50-70% sodium trifluoromethanesulfinate (CF₃SO₂Na).

3. Method according to claim 2 wherein the commercial grade sodium trifluoromethanesulfinate (CF₃SO₂Na) is pre-treated before use in step a).

4. Method according to claim 3 wherein the commercial grade sodium trifluoromethanesulfinate (CF₃SO₂Na) is pre-treated before use in step a) by slurrying in ethyl acetate, filtration and concentration.

5. Method according to claim 4 wherein the commercial grade sodium trifluoromethanesulfinate (CF₃SO₂Na) is pre-treated before use in step a) by slurrying the commercial grade sodium trifluoromethanesulfinate (CF₃SO₂Na) in ethyl acetate, heating of the resulting suspension to about 40-50° C., stirring at this temperature, filtering of the suspension, adding water to the filtrate and removing substantially all ethyl acetate.

6. Method according to claim 1 wherein the organic peroxide used in step a) is tert-butyl hydroperoxide (TBHP).

7. Method according to claim 6 wherein the organic peroxide used in step a) is tert-butyl hydroperoxide (TBHP), wherein the aqueous solution of tert-butyl hydroperoxide (TBHP) has a content of about 70% tert-butyl hydroperoxide (TBHP).

8. Method according to claim 1 wherein the organic peroxide used in step a) is continuously dosed to the reaction mixture.

9. Method according to claim 1 wherein the organic peroxide used in step a) is used in an amount of about 4 eq. in relation to uracil.

10. Method according to claim 1 wherein the addition rate of the organic peroxide used in step a) is controlled to keep the reaction temperature in a range of about 45-75° C. during addition.

11. Method according to claim 1 wherein the reaction temperature in step a) is kept in a range of about 40-100° C. after the addition of an aqueous solution of organic peroxide until the ratio of uracil:5-trifluoromethyl-uracil is equal to or below 3:97.

12. Method according to claim 1 wherein the reaction temperature in step a) is kept in a range of about 40-80° C. after the addition of an aqueous solution of organic peroxide.

13. Method according to claim 12 wherein the reaction temperature in step a) is kept in a range of about 45-75° C. after the addition of an aqueous solution of organic peroxide.

14. Method according to claim 13 wherein the reaction temperature in step a) is kept in a range of about 60-70° C. after the addition of an aqueous solution of organic peroxide.

15. Method according to claim 13 wherein the reaction temperature in step a) is kept in a range of about 45-60° C. after the addition of an aqueous solution of organic peroxide.

16. Method according to claim 1 wherein the reaction in step a) is carried out in the presence of a transition metal catalyst.

17. Method according to claim 16 wherein the reaction in step a) is carried out in the presence of FeSO₄ as catalyst.

18. Method according to claim 1 wherein the reaction in step a) is carried out in the presence of silica gel.

19. Method according to claim 1 wherein a solvent selected from among THF, 2-MeTHF, ethyl acetate and isopropyl acetate is used to extract 5-TFU obtained in reaction step a).

20. Method according to claim 1 wherein 5-TFU obtained in step a) is isolated without extraction by concentration of the aqueous phase and filtration.

21. A method for preparing 2,4-dichloro-5-trifluoromethyl-pyrimidine (5-TFP) comprising the method of claim 1 and further comprising
b) reacting 5-trifluoromethyluracil (5-TFU) with phosphoryl chloride (POC1₃) to form 2,4-dichloro-5-trifluoromethyl-pyrimidine (5-TFP).

22. Method according to claim 21 wherein the intermediate 5-TFU obtained in step a) is chlorinated with or without isolation of 5-TFU.

23. Method according to claim 22 wherein the intermediate 5-TFU obtained in step a) is chlorinated with or without isolation of 5-TFU to 5-TFP in step b) using a mixture of phosphoric acid (H₃PO₄), phosphoryl chloride (POCl₃) and diisopropylethyl amine (DIPEA).
